(19) United States
(12) Patent Application Publication
(10) Pub. No.: US 2007/0202182 A1
(43) Pub. Date: Aug. 30, 2007

(54) Preparing solid formulation of nanoparticles of pharmaceutical ingredient, including at least micron-sized particles

(76) Inventors: Kevin M. Kane, Ft. Lauderdale, FL (US); Isaac Farr, Corvallis, OR (US); Iddys D. Figueroa, Aguadilla, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/363,129

(22) Filed: Feb. 26, 2006

(51) Int. Cl. A61K 9/16; B29C 39/10; A61K 31/704; A61K 31/405; A61K 31/17; A61K 9/50

(52) U.S. Cl. 424/490; 514/26; 514/179; 514/412; 514/594; 264/4

(57) ABSTRACT

A slurry is prepared in which nanoparticles of a pharmaceutical ingredient have been formed within a solvent. A solid formulation of the nanoparticles of the pharmaceutical ingredient is prepared from the slurry, without employing electrostatic collection. The solid formulation of the nanoparticles includes at least micron-sized particles.

### BACKGROUND

In order for a drug to achieve its desired result, it typically has to be delivered to a biological site of interest. Most drugs in use today are solid ingestibles. For these drugs to be absorbed into the bloodstream and transported to a biological site of interest, they usually have to first be dissolved and then permeate the intestinal walls. The preparation of small particles can increase the dissolution rate and potentially the bioavailability of a selected drug candidate. Solubility may be modified by physically grinding a drug to yield micron size and smaller particles. However, this mechanical approach can cause chemical or physical degradation of the drug, by shearing and heat stress.

Recent advances in drug formulation have enabled drugs to be rendered in nanoparticle-size. For instance, the patent application “Nanoparticle formation of pharmaceutical ingredients,” filed on Jan. 13, 2006, and assigned Ser. No. 11/332,131 [attorney docket 200504581-1], describes multiple approaches for forming nanoparticles of drugs. However, most pharmaceutical processing equipment is geared towards micron-sized particles. Therefore, such existing equipment may then not be able to be used on nanoparticles of drugs, slowing the adoption of nanoparticle-sized drug formulations. For these and other reasons, there is a need for the present invention.

### BRIEF DESCRIPTION OF THE DRAWINGS

The drawings referenced herein form a part of the specification. Features shown in the drawing are meant as illustrative of only some embodiments of the invention, and not of all embodiments of the invention, unless otherwise explicitly indicated.

FIG. 1 is a flowchart of a method for preparing a solid formulation of nanoparticles of a pharmaceutical ingredient, which includes at least micron-sized particles, and also for subsequently processing these micron-sized particles, according to an embodiment of the invention.

FIG. 2 is a diagram illustratively depicting resulting performance of one of the parts of the method of FIG. 1, according to an embodiment of the invention.

FIG. 3 is a flowchart of a method for preparing a solid formulation of nanoparticles of a pharmaceutical ingredient, the solid formulation including at least micron-sized particles, according to an embodiment of the invention.

FIG. 4 is a diagram illustratively depicting the performance of the method of FIG. 3, according to an embodiment of the invention.

FIG. 5 is a flowchart of a method for preparing a solid formulation of nanoparticles of a pharmaceutical ingredient, the solid formulation including at least micron-sized particles, according to another embodiment of the invention.

FIG. 6 is a diagram illustratively depicting the performance of the method of FIG. 5, according to an embodiment of the invention.

FIG. 7 is a flowchart of a method for preparing a solid formulation of nanoparticles of a pharmaceutical ingredient, the solid formulation including at least micron-sized particles, according to still another embodiment of the invention.

FIG. 8 is a diagram illustratively depicting the performance of the method of FIG. 7, according to an embodiment of the invention.

### DETAILED DESCRIPTION OF THE DRAWINGS

In the following detailed description of exemplary embodiments of the invention, reference is made to the accompanying drawings that form a part hereof, and in which is shown by way of illustration specific exemplary embodiments in which the invention may be practiced. These embodiments are described in sufficient detail to enable those skilled in the art to practice the invention. Other embodiments may be utilized, and logical, mechanical, and other changes may be made without departing from the spirit or scope of the present invention. The following detailed description is, therefore, not to be taken in a limiting sense, and the scope of the present invention is defined only by the appended claims.

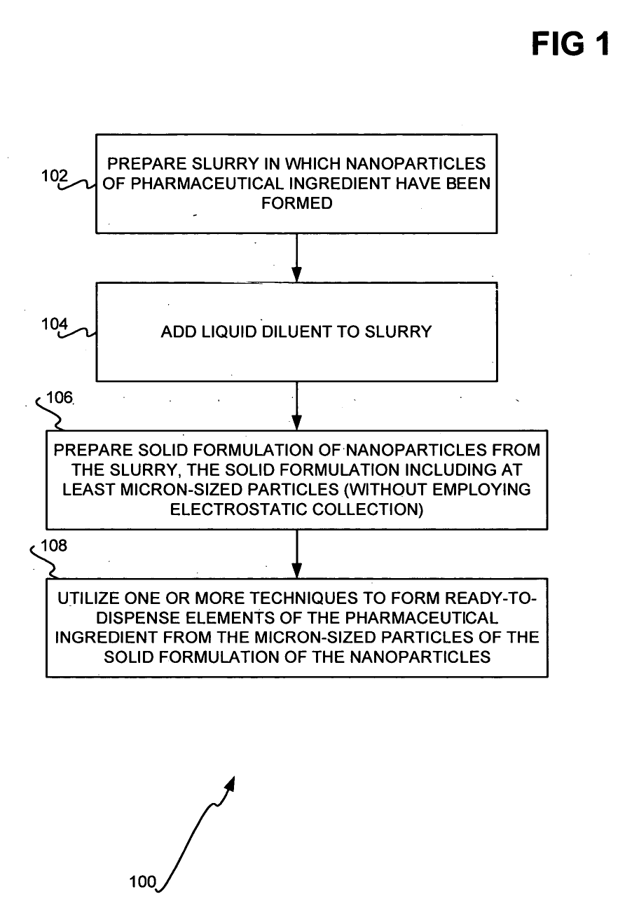

FIG. 1 shows a method 100 for preparing a solid formulation of nanoparticles of a pharmaceutical ingredient, which includes at least micron-sized particles, and also for subsequently processing these micron-sized particles, according to an embodiment of the invention. The pharmaceutical ingredient may be an active pharmaceutical ingredient, such as glyburide, prednisolone, or indomethacin, among other types of active pharmaceutical ingredients. Other types of pharmaceutical ingredients, for instance, include betamethasone acetate, triamcinolone acetonide, piroxicam, glimepiride, glipizide, and digoxin.

First, a slurry is prepared (102), in which nanoparticles of a pharmaceutical ingredient have been formed within a solvent. In one embodiment, one or more of the approaches described in the patent application “Nanoparticle formation of pharmaceutical ingredients,” filed on Jan. 13, 2006, and assigned Ser. No. 11/332,131 [attorney docket 200504581-1], may be employed to prepare this slurry. The solvent may be a single solvent, or a multiple solvent, such as a binary solvent.

For instance, the solvent may be a binary solvent, such as ethanol:chloroform having a proportion of 80% ethanol to 20% chloroform by volume (i.e., 80% of the volume of the solvent is ethanol, and 20% of the volume is chloroform). Other solvent combinations have also been proven to result in nanoparticle formation. These include 80% ethanol by volume and 20% water by volume; 80% methanol by volume and 20% water by volume; and, 80% acetone by volume and 20% water by volume. It is noted that the terminology “solvent” as used herein is inclusive of the plural “solvents,” when, for instance, a binary solvent, or another type of multiple solvent, like a ternary solvent, is used.

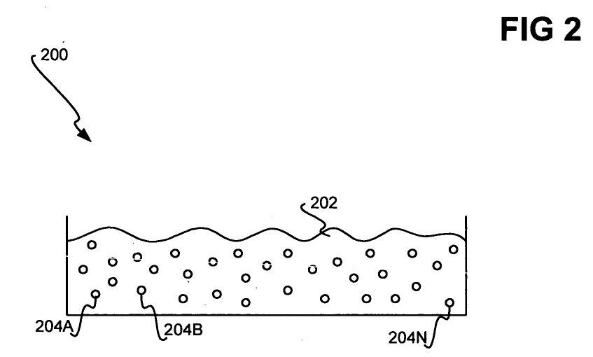

FIG. 2 illustratively depicts the results of the performance of part 102 of the method 100, according to an embodiment of the invention. A slurry 200 includes a solvent 202. Within the solvent are a number of nanoparticles of a pharmaceutical ingredient 204A, 204B, . . . , 204N. These nanoparticles are collectively referred to as the nanoparticles 204. In one embodiment, the slurry 200 may include a residual dissolved portion of the pharmaceutical ingredient that has not formed into the nanoparticles 204, which is not shown in FIG. 2.

Referring back to FIG. 1, in one embodiment a liquid diluent may be added to the slurry that has been prepared (104). This liquid diluent may be water, more of the solvent, or another type of liquid diluent. Adding the liquid diluent decreases the concentration of the nanoparticles relative to other portions of the slurry, such as the solvent and the liquid diluent, and thus dilutes the nanoparticles within the slurry.

Next, a solid formulation of the nanoparticles is prepared from the slurry (106). This solid formulation includes at least micron-sized particles formed at least from the nanoparticles of the pharmaceutical ingredient. The micron-sized particles are thus at least one micron in size, whereas the nanoparticles are less than one micron in size, and may be only nanometers in size. Different techniques and approaches for preparing such a solid formulation of the nanoparticles, resulting in micron-sized particles, are described in detail later in the detailed description.

It is noted that electrostatic collection is not employed in preparing the solid formulation of the nanoparticles from the slurry, in at least some embodiments of the invention. Electrostatic collection can be a complex procedure, and not having to employ such electrostatic collection, such as by instead using one of the techniques described in detail later in the detailed description, simplifies the solid formulation preparation.

Finally, one or more techniques may be utilized to form ready-to-dispense elements, such as pills, of the pharmaceutical ingredient from the micron-sized particles of the solid formulation of the nanoparticles of the pharmaceutical ingredient (108). Such techniques can include spray-drying, fluidized bed coating, dry and wet milling, dry blending, and compaction, among other types of techniques. These techniques are employable within the context of micron-sized particles, and rely upon the containment of stray particles using high efficiency particulate air (HEPA) filtering systems, which can screen only micron-sized particles, and not, for instance, nanoparticles.

Therefore, embodiments of the invention are advantageous, because they allow for pharmaceutical ingredients to have increased bioavailability, as a result of the nanoparticles, but provide for ready-to-dispense elements to be formed, as a result of the micron-sized particles formed from the nanoparticles. That is, embodiments of the invention allow for the utilization of industry-standard pharmaceutical processing equipment, which is optimized for micron-sized particles, in relation to nanoparticles of pharmaceutical ingredients. As a result, embodiments of the invention facilitate the adoption of nanoparticles into drug formulations, since existing pharmaceutical processing equipment can be employed in relation to such nanoparticles.

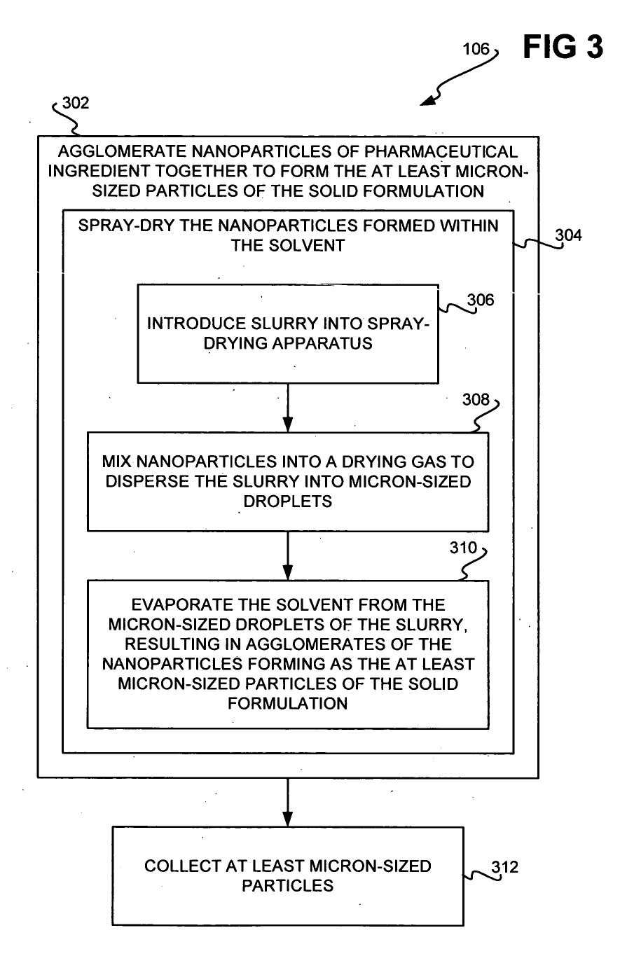

FIG. 3 shows a method for preparing the solid formulation of nanoparticles from the slurry, including at least micron-sized particles, in part 106 of the method 100 of FIG. 1, according to an embodiment of the invention. In particular, the nanoparticles of the pharmaceutical ingredient are agglomerated together to form the at least micron-sized particles of the solid formulation (302). In one embodiment, the nano-particles within the solvent may be spray-dried to result in their agglomeration as the at least micron-sized particles of the solid formulation (304).

Spray-drying of the nanoparticles within the solvent of the slurry can be accomplished within part 304 as follows. First, the slurry is introduced into a spray-drying apparatus (306). Next, the nanoparticles are mixed into a drying gas, to disperse the slurry into micron-sized droplets (308). The temperature and relative humidity of the flow of the drying gas may be appropriately controlled to result in dispersal of the slurry into micron-sized droplets. The drying gas may be compressed air, nitrogen, carbon dioxide, or another type of drying gas that has little or no chemical reactivity with the material being dried.

The solvent is evaporated from the micron-sized droplets of the slurry (310), resulting in agglomerates of the nanoparticles forming as the at least micron-sized particles of the solid formulation. The introduction, mixing, and evaporation of parts 306, 308, and 310 thus results in the concentration or aggregation of the nanoparticles of the slurry into larger, micron-sized agglomerates that retain the high surface area-to-volume ratio of the nanoparticles. However, the nanoparticle agglomerates (i.e., the micron-sized particles) have a sufficiently high density. As a result, the micron-sized particles can be collected (312), using existing equipment, such as a cyclone-collecting apparatus.

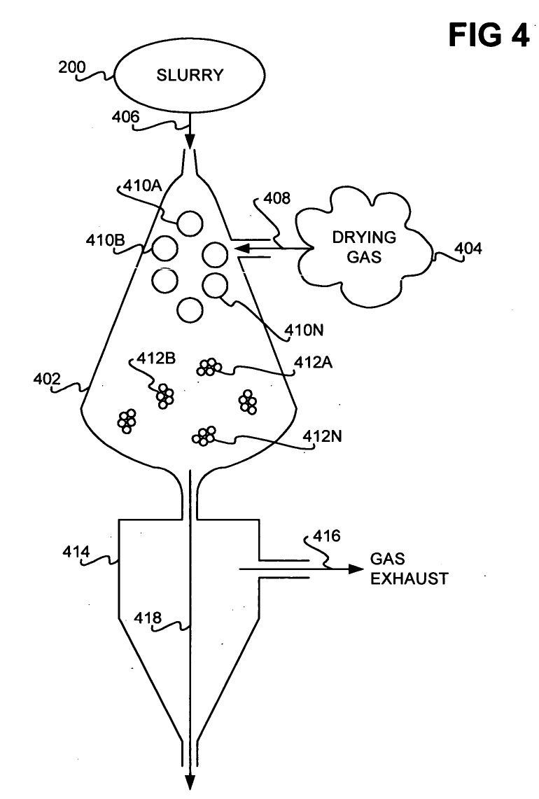

FIG. 4 illustratively depicts the performance of the solid formulation preparation and collection of the embodiment of FIG. 3, according to an embodiment of the invention. There is a spray-drying apparatus 402, as well as a cyclone-collecting apparatus 414. The slurry 200 is introduced into the spray-drying apparatus 402, as indicated by the arrow 406. As the slurry 200 descends through the spray-drying apparatus 402, the nanoparticles of the slurry 200 are mixed into a drying gas 404, which is also introduced into the spray-drying apparatus 402, as indicated by the arrow 408.

The resulting of the mixing of the nanoparticles of the slurry 200 into the drying gas 404 is the dispersion of the slurry 200 into micron-sized droplets 410A, 410B, . . . , 410N, collectively referred to as the droplets 410. The solvent from these droplets 410 evaporates, resulting in the formation of agglomerates 412A, 412B, . . . , 412N, collectively referred to as the agglomerates 412, of the nanoparticles. These agglomerates 412 are the at least micron-sized particles of the solid formulation of the pharmaceutical ingredient.

The drying gas 404, and the evaporated solvent, are exhausted from the cyclone-collecting apparatus 414, as indicated by the arrow 416. However, the micron-sized particles themselves are collected within the cyclone-collecting apparatus 414, as indicated by the arrow 418. The collected micron-sized particles can be subjected to various techniques to form ready-to-dispense elements of the pharmaceutical ingredient, as has been described in relation to part 108 of the method 100 of FIG. 1.

It is noted that the slurry 200 can include a residual dissolved portion of the pharmaceutical ingredient that has not been formed into nanoparticles, as has been described. In this case, the evaporation of the solvent in part 310 of FIG. 3 can result in precipitation of this residual dissolved portion of the pharmaceutical ingredients onto the nanoparticles. As a result, the micron-sized particles that are formed include both such precipitation, as well as agglomeration of nanoparticles.

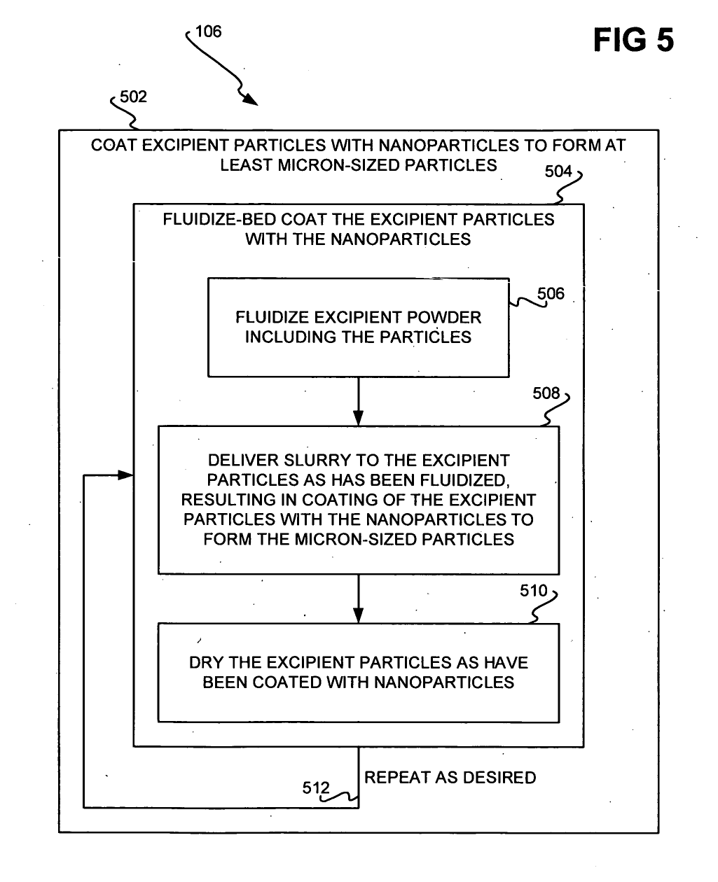

FIG. 5 shows a method for preparing the solid formulation of nanoparticles from the slurry, including at least micron-sized particles, in part 106 of the method 100 of FIG. 1, according to another embodiment of the invention. In particular, excipient particles are coated with nanoparticles to form the at least micron-sized particles (502). In one embodiment, the excipient particles may be fluidize-bed coated with the nanoparticles to result in the micron-sized particles of the solid formulation (504).

Fluidize-bed coating of the excipient particles with the nanoparticles formed within the slurry can be accomplished within part 504 as follows. First, an excipient powder, which includes the excipient particles, is fluidized (506). For instance, the excipient powder may be introduced into a fluidized bed-coating apparatus. Next, the slurry is delivered to the fluidized excipient particles (508), resulting in coating of the excipient particles with the nanoparticles formed within the slurry, to form the micron-sized particles. Thus, an atomizing-spraying apparatus may be used to deliver the slurry, including the nanoparticles formed therewithin, to the excipient particles of the powder.

The slurry may be top-sprayed onto the excipient particles, bottom- or fountain-sprayed onto the excipient particles, or tangential- or side-sprayed onto the excipient particles. The excipient particles may be a water-insoluble material or a water-soluble material. For instance, the excipient particles may be cellulose acetate, a polyacrylic-based excipient, fumed silica, talc, titanium dioxide, zinc oxide, cornstarch, hydroxypropyl methylcellulose (HPMC), or another type of excipient particle. In another embodiment, the excipient particles themselves may be of the same or different pharmaceutical ingredient as the nanoparticles that have been formed within the slurry. That is, the excipient parts may have the same or different chemical composition as that of the pharmaceutical ingredient of the nanoparticles in such an embodiment. Furthermore, the excipient particles may themselves be at least micron-sized, such that the micron-sized particles of the solid formulation of the pharmaceutical ingredient result from coating the nanoparticles of the pharmaceutical ingredient onto these excipient particles.

Thereafter, the excipient particles, as have been coated with the nanoparticles of the pharmaceutical ingredient, are dried (510). For instance, a drying gas may be introduced into the fluidized bed-coating apparatus to dry the excipient particles as coated. The fluidize bed-coating process of part 504 may be repeated as desired (512), to achieve the needed, desired, or appropriate coating of the excipient particles with the nanoparticles of the pharmaceutical ingredient. Thus, the excipient powder fluidized in part 506 is that which has had its excipient particles already coated with nanoparticles, more of the slurry with more of the nanoparticles is introduced in part 508, and then the excipient particles as twice (or more) coated with the nanoparticles are dried in part 510.

During the fluidized-bed coating process in part 504, the flow rates of the slurry and of a fluidizing gas used to fluidize the excipient powder can be controlled to affect the thickness of nanoparticle coating on the excipient particles. Furthermore, the temperature of the fluidizing gas, or of a separate drying gas if a different gas is used for drying, may be controlled to optimize drying of the excipient particles after coating with nanoparticles. Examples of fluidizing gases include compressed air, nitrogen, carbon dioxide, as well as other types of fluidizing gases. Thus, the fluidizing gas can serve as the drying gas to dry the nanoparticle-coated excipient particles, or a different drying gas can be employed. Examples of drying gases that are separate from the fluidizing gas include compressed air, nitrogen, carbon dioxide, as well as other types of drying gases.

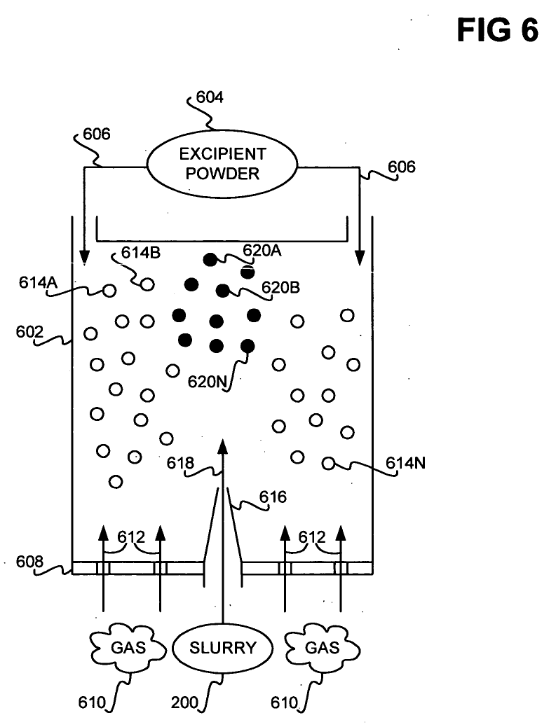

FIG. 6 illustratively depicts the performance of the solid formulation preparation of the embodiment of FIG. 5, according to an embodiment of the invention. There is a fluidized bed-coating apparatus 602, and an atomizing-spraying apparatus 616. Excipient powder 604 is introduced into the fluidized bed-coating apparatus 602, as indicated by the arrows 606. A fluidizing gas 610 is introduced through a porous plate 608 at the bottom of the fluidized bed-coating apparatus 602, as indicated by the arrows 612. The fluidizing gas 610 fluidizes the excipient powder 604 into a number of fluidized excipient particles 614A, 614B, . . . , 614N, collectively referred to as the fluidized excipient particles 614.

Next, the slurry 200 is delivered to the excipient powder 604, as has been fluidized as the excipient particles 614, via the atomizing-spraying apparatus 616, as indicated by the arrow 618. The atomizing-spraying apparatus 616 atomizes the slurry 200, and sprays the slurry 200 onto the excipient particles 614. The result is the pharmaceutical ingredient nanoparticle-coated excipient particles 620A, 620B, . . . , 620N, collectively referred to as the nanoparticle-coated excipient particles 620. That is, the atomized slurry 200 coats the particles 614, such that the nanoparticles coat the particles 614, to result in the nanoparticle-coated particles 620. These nanoparticle-coated particles 620 are at least micron-sized particles, where the excipient particles 614 themselves are at least micron-sized.

It is noted that the atomizing-spraying apparatus 616 as depicted in the example of FIG. 6 is a bottom or fountain sprayer, which is also referred to as a Wurster-configuration sprayer. In other embodiments, a top sprayer, or a tangential or side sprayer may be employed. Once the nanoparticles of the slurry 200 have coated the excipient particles 614, resulting in the nanoparticle-coated excipient particles 620, the fluidizing gas 610 also serves to dry the coated excipient particles 620. In another embodiment, however, a separate drying as may be introduced, in addition to the fluidizing gas 610, to dry the coated excipient particles 620. The at least micron-sized particles, as the nanoparticle-coated excipient particles, can then be subjected to various techniques to form ready-to-dispense elements of the pharmaceutical ingredient, as has been described in relation to part 108 of the method 100 of FIG. 1.

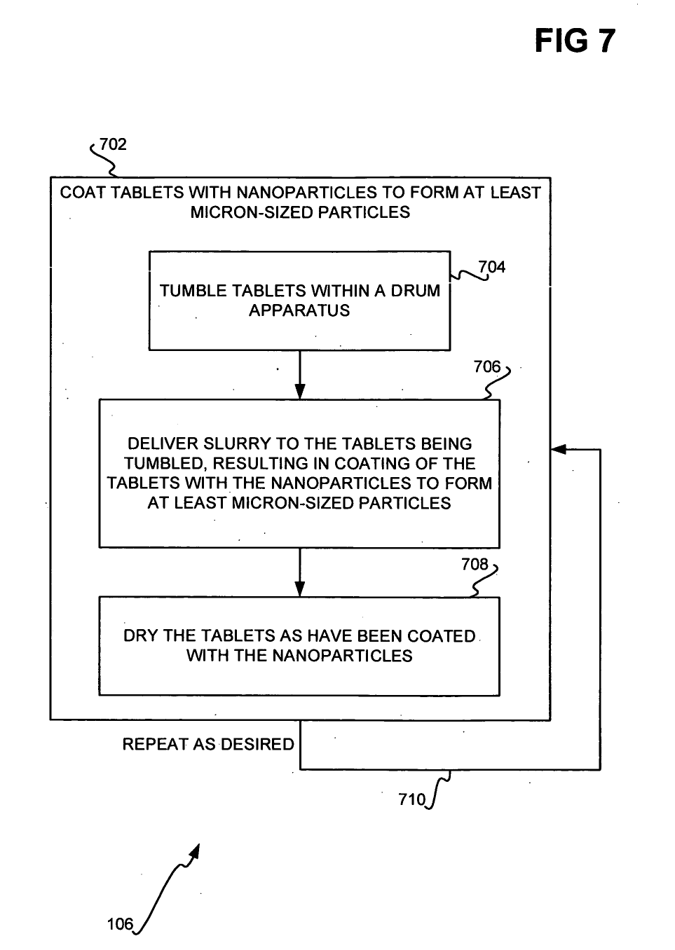

FIG. 7 shows a method for preparing the solid formulation of nanoparticles from the slurry, including at least micron-sized particles, in part 106 of the method 100 of FIG. 1, according to still another embodiment of the invention. In particular, tablets are coated with nanoparticles to form the at least micron-sized particles (702). The tablets may be a water-insoluble material or a water-soluble material. For instance, the tablets may be comprised substantially of cellulose acetate, a polyacrylic-based excipient, fumed silica, talc, titanium dioxide, zinc oxide, cornstarch, hydroxypropyl methylcellulose (HPMC), or another type of material. In another embodiment, the tablets may themselves be comprised substantially of a pharmaceutical ingredient, having the same or different chemical composition as that of the pharmaceutical ingredient of the nanoparticles. Furthermore, the tablets particles are at least micron-sized, such that the micron-sized particles of the solid formulation of the pharmaceutical ingredient result from coating the nanoparticles of the pharmaceutical ingredient onto these tablets.

In one embodiment, coating the tablets with the nanoparticles formed within the slurry can be accomplished within part 702 as follows. First, the tablets are tumbled within a drum apparatus (704). Next, the slurry is delivered to the tablets being tumbled (706), resulting in coating of the tablets with the nanoparticles formed within the slurry, to form the micron-sized particles. The slurry may be delivered via utilization of an atomizing-spraying apparatus. Thereafter, the tablets, as have been coated with the nanoparticles of the pharmaceutical ingredient, are dried (708). For instance, a drying gas may be introduced into the drum apparatus to dry the tablets as coated.

The coating process of part 702 may be repeated as desired (710), to achieve the needed, desired, or appropriate coating of the tablets with the nanoparticles of the pharmaceutical ingredient. Thus, the tablets tumbled in part 704 are that which have already been coated with nanoparticles. Likewise, more of the slurry with more of the nanoparticles is introduced in part 706, and then the tablets as twice (or more) coated with the nanoparticles are dried in part 708.

During the coating process in part 702, the flow rates of the slurry and of a drying gas can be controlled to affect the thickness of the nanoparticle coating on the tablets. Furthermore, the temperature of the drying gas may be controlled to optimize drying of the tablets after coating with nanoparticles. Examples of drying gases include compressed air, nitrogen, carbon dioxide, as well as other types of drying gases.

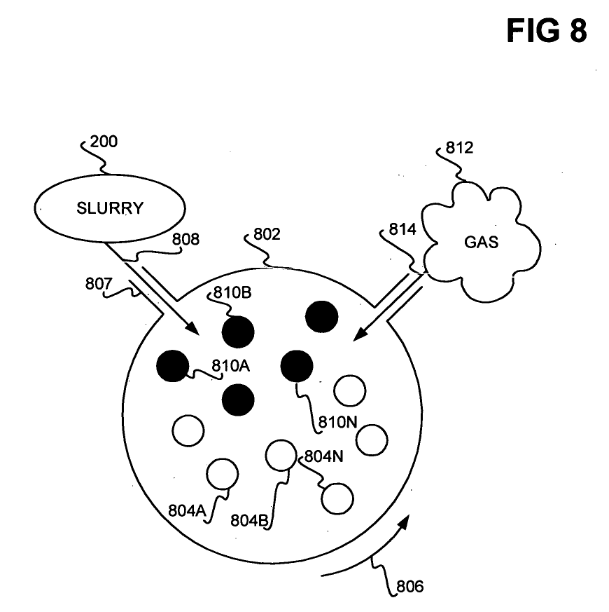

FIG. 8 illustratively depicts the performance of the solid formulation preparation of the embodiment of FIG. 7, according to an embodiment of the invention. There is a drum apparatus 802, and an atomizing-spraying apparatus 807. Tablets 804A, 804B, . . . , 804N, collectively referred to as the tablets 804, are tumbled within the drum apparatus 802, such as via rotation of the drum apparatus 802, as indicated by the arrow 806. The slurry 200 is delivered to the tablets 804, via the atomizing-spraying apparatus 807, as indicated by the arrow 808. The atomizing-spraying apparatus 807 atomizes the slurry 200, and sprays the slurry 200 onto the tablets 804.

The result is the pharmaceutical ingredient nanoparticle-coated tablets 810A, 810B, . . . , 810N, collectively referred to as the nanoparticle-coated tablets 810. That is, the atomized slurry 200 coats the tablets 804, such that the nanoparticles coat the tablets 804, to result in the nanoparticle-coated tablets 810. The nanoparticle-coated tablets 810 are the at least micron-sized particles. Further, a drying gas 812 is introduced into the drum apparatus 802, as indicated by the arrow 814, to dry the coated tablets 810. The at least micron-sized particles, as the coated tablets 810, can then be subjected to various techniques to form ready-to-dispense elements of the pharmaceutical ingredient, as has been described in relation to part 108 of the method 100 of FIG. 1.

It is noted, therefore, that although specific embodiments have been illustrated and described herein, it will be appreciated by those of ordinary skill in the art that any arrangement calculated to achieve the same purpose may be substituted for the specific embodiments shown. This application is intended to cover any adaptations or variations of the disclosed embodiments of the present invention. It is thus manifestly intended that this invention be limited only by the claims and equivalents thereof.

We claim:

1. A method comprising:
providing a slurry in which a plurality of nanoparticles of a pharmaceutical ingredient have been formed within a solvent; and,
preparing a solid formulation of the nanoparticles of the pharmaceutical ingredient from the slurry, the solid formulation of the nanoparticles comprising a plurality of at least micron-sized particles, without employing electrostatic collection.

2. The method of claim 1, wherein preparing the solid formulation of the nanoparticles of the pharmaceutical ingredient from the slurry comprises agglomerating the nanoparticles of the pharmaceutical ingredient together to form the at least micron-sized particles.

3. The method of claim 2, wherein agglomerating the nanoparticles together to form the at least micron-sized particles comprises spray-drying the nanoparticles formed within the solvent.

4. The method of claim 1, wherein preparing the solid formulation of the nanoparticles of the pharmaceutical ingredient from the slurry comprises coating a plurality of excipient particles with the nanoparticles of the pharmaceutical ingredient to form the at least micron-sized particles.

5. The method of claim 4, wherein coating the excipient particles with the nanoparticles of the pharmaceutical ingredient to form the at least micron-sized particles comprises fluidized-bed coating the excipient particles with the nanoparticles.

6. The method of claim 1, wherein preparing the solid formulation of the nanoparticles of the pharmaceutical ingredient from the slurry comprises coating a plurality of tablets with the nanoparticles of the pharmaceutical ingredient to form the at least micron-sized particles.

7. The method of claim 1, further comprising adding a liquid diluent to the slurry prior to preparing the solid formulation of the nanoparticles of the pharmaceutical ingredient.

8. The method of claim 1, further comprising utilizing one or more techniques to form ready-to-dispense elements of the pharmaceutical ingredient from the micron-sized particles of the solid formulation of the nanoparticles of the pharmaceutical ingredient.

9. The method of claim 1, wherein the pharmaceutical ingredient is one of: glyburide, prednisolone, and indomethacin, betamethasone acetate, triamcinolone acetonide, piroxicam, glimepiride, glipizide, or digoxin.

10. A solid formulation of nanoparticles of a pharmaceutical ingredient including a plurality of at least micron-sized particles, formed by performing a method comprising:
providing a slurry in which a plurality of nanoparticles of a pharmaceutical ingredient have been formed within a solvent; and,
agglomerating the nanoparticles of the pharmaceutical ingredient together to form the at least micron-sized particles of the solid formulation.

11. The solid formulation of claim 10, wherein agglomerating the nanoparticles together to form the at least micron-sized particles comprises spray-drying the nanoparticles formed within the solvent.

12. The solid formulation of claim 11, wherein spray-drying the nanoparticles formed within the solvent comprises:
introducing the slurry into a spray-drying apparatus; and,
mixing the nanoparticles into a drying gas to disperse the slurry into micron-sized droplets; and,
evaporating the solvent from the micron-sized droplets of the slurry, resulting in agglomerates of the nanoparticles forming as the at least micron-sized particles.

13. The solid formulation of claim 12, wherein the slurry further includes a residual dissolved portion of the pharmaceutical ingredient that has not been formed into the nanoparticles of the pharmaceutical ingredient, such that evaporating the solvent from the micron-sized droplets of the slurry further results in precipitation of the residual dissolved portion of the pharmaceutical ingredient onto the nanoparticles as part of the at least micron-sized particles.

14. A solid formulation of nanoparticles of a pharmaceutical ingredient including a plurality of at least micron-sized particles, formed by performing a method comprising:
providing a slurry in which a plurality of nanoparticles of a pharmaceutical ingredient have been formed within a solvent; and,
coating a plurality of excipient particles with the nanoparticles of the pharmaceutical ingredient to form the at least micron-sized particles.

15. The solid formulation of claim 14, wherein coating the excipient particles with the nanoparticles of the pharmaceutical ingredient to form the at least micron-sized particles comprises fluidized-bed coating the excipient particles with the nanoparticles.

16. The solid formulation of claim 15, wherein fluidized-bed coating the excipient particles with the nanoparticles comprises:
fluidizing an excipient powder comprising the excipient particles to continuously mix the excipient powder;
delivering the slurry including the nanoparticles to the excipient powder as has been fluidized, resulting in coating of the excipient particles with the nanoparticles as the at least micron-sized particles; and,
drying the excipient particles as have been coated with the nanoparticles.

17. The solid formulation of claim 16, wherein fluidizing the excipient power comprises introducing the excipient powder into a fluidized bed-coating apparatus.

18. The solid formulation of claim 16, wherein delivering the slurry including the nanoparticles to the excipient powder as has been fluidized comprises using an atomizing-spraying apparatus to deliver the slurry including the nanoparticles to the excipient powder.

19. The solid formulation of claim 18, wherein using the atomizing-spraying apparatus to deliver the slurry including the nanoparticles to the excipient powder comprises one of:
top-spraying the slurry including the nanoparticles to the excipient powder;
bottom/fountain-spraying the slurry including the nanoparticles to the excipient powder; and,
tangential/side-spraying the slurry including the nanoparticles to the excipient powder.

20. The solid formulation of claim 16, wherein fluidized-bed coating the excipient particles with the nanoparticles further comprises:
fluidizing the excipient powder comprising the excipient particles as have been coated with the nanoparticles to continuously mix the excipient powder;
delivering more of the slurry including more of the nanoparticles to the excipient powder as has been fluidized, resulting in a second coating of the excipient particles with the nanoparticles as the at least micron-sized particles; and,
drying the excipient particles as have been twice coated with the nanoparticles.

21. The solid formulation of claim 16, wherein fluidized-bed coating the excipient particles with the nanoparticles further comprises delivering more of the slurry including more of the nanoparticles to the excipient powder as has been fluidized, resulting in a second coating of the excipient particles with the nanoparticles as the at least micron-sized particles.

22. The solid formulation of claim 16, wherein the excipient particles comprise one of a water-insoluble material and a water soluble material.

23. The solid formulation of claim 16, wherein the excipient particles comprise one of: cellulose acetate, a polyacrylic-based excipient, fumed silica, talc, titanium dioxide, zinc oxide, cornstarch, and hydroxypropyl methylcellulose (HPMC).

24. A solid formulation of nanoparticles of a pharmaceutical ingredient including a plurality of at least micron-sized particles, formed by performing a method comprising:
providing a slurry in which a plurality of nanoparticles of a pharmaceutical ingredient have been formed within a solvent; and,
coating a plurality of tablets with the nanoparticles of the pharmaceutical ingredient to form the at least micron-sized particles.

25. The solid formulation of claim 24, wherein coating the tablets with the nanoparticles of the pharmaceutical ingredient to form the at least micron-sized particles comprises:
tumbling the tablets within a drum apparatus;
delivering the slurry including the nanoparticles to the tablets being tumbled, resulting in coating of the tablets with the nanoparticles as the at least micron-sized particles; and,
drying the tablets as have been coated with the nanoparticles.

26. The solid formulation of claim 25, wherein delivering the slurry including the nanoparticles to the tablets being tumbled comprises using an atomizing-spraying apparatus to deliver the slurry including the nanoparticles to the tablets.

27. The solid formulation of claim 25, wherein coating the tablets with the nanoparticles of the pharmaceutical ingredient to form the at least micron-sized particles further comprises:
tumbling the tablets as have been coated with the nanoparticles within the drum apparatus;
delivering more of the slurry including more of the nanoparticles to the tablets being tumbled, resulting in a second coating of the tablets with the nanoparticles as the at least micron-sized particles; and,
drying the tablets as have been twice coated with the nanoparticles.

28. The solid formulation claim 25, wherein coating the tablets with the nanoparticles of the pharmaceutical ingredient to form the at least micron-sized particles further comprises delivering more of the slurry including more of the nanoparticles to the tablets being tumbled, resulting in a second coating of the tablets with the nanoparticles as the at least micron-sized particles.

29. The solid formulation of claim 24, wherein the tablets comprise one of a water-insoluble material and a water-soluble material.

30. The solid formulation of claim 24, wherein the tablets comprise one of: cellulose acetate, a polyacrylic-based excipient, fumed silica, talc, titanium dioxide, zinc oxide, cornstarch, and hydroxypropyl methylcellulose (HPMC).

31. The solid formulation of claim 24, wherein the tablets comprise a pharmaceutical ingredient, the pharmaceutical ingredient of the tablets having an identical chemical composition as that of the pharmaceutical ingredient of the nanoparticles.

32. The solid formulation of claim 24, wherein the tablets comprise a pharmaceutical ingredient, the pharmaceutical ingredient of the tablets having a different chemical composition as that of the pharmaceutical ingredient of the nanoparticles.
